(19) United States
(12) Patent Application Publication
(10) Pub. No.: US 2010/0076306 A1
(43) Pub. Date: Mar. 25, 2010

(54) OPTICAL CAMERA CALIBRATION FOR CAS NAVIGATION

(76) Inventors: Emmanuel Daigneault, Montreal (CA); Ahmed Aoude, Pointe-Claire (CA)

Correspondence Address: OGILVY RENAULT LLP, 1, Place Ville Marie, SUITE 2500, MONTREAL, QC, H3B 1R1, CA

(21) Appl. No.: 12/564,494

(22) Filed: Sep. 22, 2009

Related U.S. Application Data
(60) Provisional application No. 61/100,174, filed on Sep. 25, 2008.

(51) Int. Cl. A61B 5/05

(52) U.S. Cl. 600/426

(57) ABSTRACT

A navigation system for tracking an object during surgery, comprises markers adapted to be connected to an object in a known relation. An optical camera detects a light signal from the at least one marker, the optical camera generating tracking data associated with a position of the markers from the light signal. A transparent shield is positioned between the optical camera and the markers during surgery to allow the optical camera to be within the sterile field. Calibration data is associated with an effect of the shield on the light signal passing therethrough to be detected by the optical camera. A position and orientation calculator calculates a position and/or orientation of the object as a function of the tracking data, of the calibration data, and of the known relation between the object and the markers. A method is provided for tracking an object in computer-assisted surgery.

### CROSS-REFERENCE TO RELATED APPLICATION

The present patent application claims priority on U.S. Patent Application No. 61/100,174, filed on Sep. 25, 2008.

### FIELD OF THE APPLICATION

The present application relates to optical cameras used for navigation in computer-assisted surgery, and more particularly to their use within sterile fields.

### BACKGROUND OF THE ART

Optical tracking cameras are commonly used in computer-assisted surgery (CAS) systems as part of navigation systems. The optical tracking cameras detect the position of markers, and will generate from the position of the markers surgical data, such as the position of bones and tools, axes, angles, models, etc.

The markers used with navigation systems may be active or passive markers. An active marker typically uses LEDs emitting infrared light, which light is detected by the optical camera. A passive marker is made of a material that retro reflects infrared light emitted by the optical camera, back toward the optical camera for detection.

The detected light represents position data that is transferred to a processing unit of the navigation system for determining a position of the marker, whereby a position and/or orientation of a tool or a bodily element may be calculated, amongst numerous other possibilities.

Northern Digital Inc. has developed an optical camera (i.e., optical measurement system) commonly used with navigation systems. One series of products used in computer-assisted surgery is called Polaris™. One issue associated with navigation in computer-assisted surgery is the accuracy of the data. For instance, in orthopedic surgery, alterations are performed on bones, whereby the navigational data must be accurate considering that it is used to determine bone cutting lengths and angles, provide models, amongst numerous other possibilities. In view of the accuracy requirements, optical cameras are calibrated before use with a coordinate measuring machine (CMM), so as to ensure their measurements are within industry standards.

Moreover, when used in computer-assisted surgery, optical cameras must either be positioned outside the sterile field, or must be sterilized, so as to avoid any contamination of a patient. Considering the issues involved with the sterilization of complex electronic equipment, the optical cameras are commonly positioned outside the sterile field. The optical cameras must therefore cover an increased field of view due to the distance by which it is separated from the sterile field. Accordingly, optical cameras of large size are used, which cameras are cumbersome and difficult to move around. An example is the difference in the measurement volumes between the Vicra™ and Spectra™ models of the Polaris™ line. The Spectra™ model tracks within a greater measurement volume, but is larger and heavier, and therefore not as versatile. The Vicra™ model on the other hand is smaller and easier to displace, but must be closer to the surgical table as it has a smaller measurement volume, and therefore may be within the sterile field.

### SUMMARY OF THE APPLICATION

It is therefore an aim of the present disclosure to provide an optical camera configuration and navigation system that address issues associated with the prior art.

It is a further aim of the present disclosure to provide a method for navigating with the optical camera configuration.

Therefore, in accordance with the present application, there is provided a navigation system for tracking an object in a sterile field during surgery, comprising: at least one marker adapted to be connected to the object in a known relation, the object being used in the sterile field during surgery; an optical camera detecting a light signal from the at least one marker, the optical camera generating tracking data associated with a position of the at least one marker from the light signal; a shield on the optical camera, the shield being positioned between the optical camera and the at least one marker during surgery to allow at least a portion of the optical camera to be within the sterile field; calibration data associated with an effect of the shield on the light signal passing therethrough to be detected by the optical camera; and a position and orientation calculator for calculating at least one of a position and an orientation of the object as a function of the tracking data, of the calibration data, and of the known relation between the object and the at least one marker.

Further in accordance with the present application, there is provided a method for tracking an object in computer-assisted surgery, comprising: installing a shield on at least a portion of an optical camera, the shield affecting a light signal received by the optical camera; positioning at least a portion of the optical camera within a sterile field with the object being in the sterile field and the shield being between the optical camera and the object; and tracking the object as a function of a light signal from the object received by the optical camera after passing through the shield.

### BRIEF DESCRIPTION OF THE DRAWINGS

FIG. 1 is a schematic assembly view of an optical camera with a sterile-zone shield in accordance with an exemplary embodiment of the present disclosure;

FIG. 2 is a block diagram of a navigation system as used with the optical camera and the sterile-zone shield of FIG. 1; and

FIG. 3 is a method for tracking objects with the optical camera and the sterile-zone shield of FIG. 1.

### DESCRIPTION OF THE EXEMPLARY EMBODIMENTS

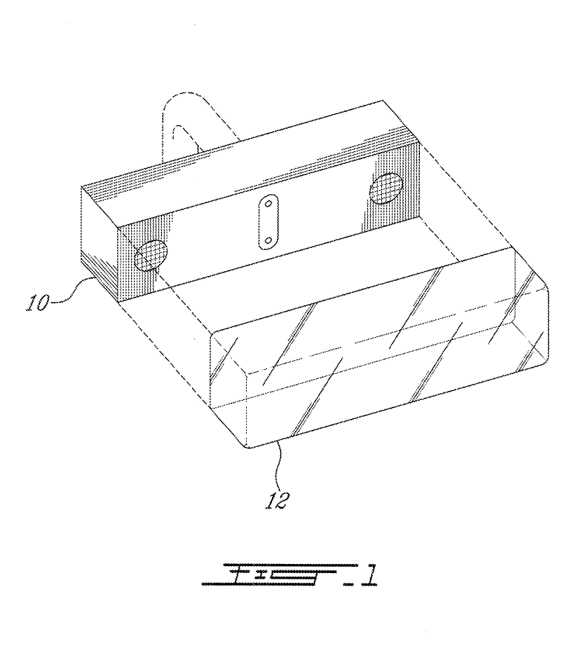

Referring to the drawings and more particularly to FIG. 1, an optical camera 10 (i.e., sensor) has a head featuring at least a pair of sensors and an infrared emitter, and is mounted to a post, amongst numerous other possible embodiments. The optical camera 10 is of the type used either with active or passive markers, or hybrid for being used with both active and passive markers. The optical camera 10 therefore has the option of emitting light.

A sterile-zone shield 12 is releasably secured to the optical camera 10, so as to form a shield between the patient in the sterile zone and the optical camera 10.

According to an embodiment, the shield 12 is made of a see-through polymeric material (e.g., transparent). The shield 12 is made of a sterile material, and all necessary precautions are taken during transportation and handling of the shield 12 so as not to contaminate the shield 12 prior to use. The shield 12 may be sold or supplied to the surgery room as a “single-use sterile device” (e.g., sold sterile in appropriate disposable sterile packaging).

As is shown in FIG. 1, the shield 12 is molded so as to conform to the shape of the head of the camera 10 in a known manner. According to this known manner, all factors affecting the light passing through the shield 12 are known, as well as their effect on the light (e.g., diffusion, diffraction). In this known manner, the distance between the sensors of the optical camera 10 and the shield 12 is within a known range of values. Also, when pieces are molded in polymer, the density of the polymer is generally within an acceptable range of densities. As an alternative to molding, the shield 12 may be manufactured by any other suitable manufacturing process, as long as its placement on the optical camera 10 is repeatable, and as long as it is repeatable in terms of the effect of diffraction on the optical camera 10.

Although the configuration of optical camera 10 and shield 12 illustrated in FIG. 1 shows a shield shaped to match the shape of the head of the optical camera 10, it is considered to have a shield 12 covering all of the optical camera 10 (i.e., including the stand). In the embodiment of FIG. 1, the camera 10 is of the type having a rearwardly projecting stand, whereby it is possible to position the stand outside of the sterile field while the head is inside. However, it is considered to place all of the optical camera 10 within the sterile field, with an appropriate shield 12 fully covering the front of the stand as well, in similar fashion to the shield 12 of FIG. 1.

Although the molded shield 12 is an exemplary embodiment, it is considered to provide shielding in different configurations. For example, the shield 12 may be a sheet that wraps around the camera 10. In another embodiment, the shield 12 comprises panels forming a see-through screen between the camera 10 and the surgical table. In both these configurations and other configurations, the interconnection between the shield 12 and the camera 10 must be predictable and repeatable, as the manner in which the shield 12 alters the light signal received by the camera 10 must be constant in view of a calibration of the camera 10 with the shield 12.

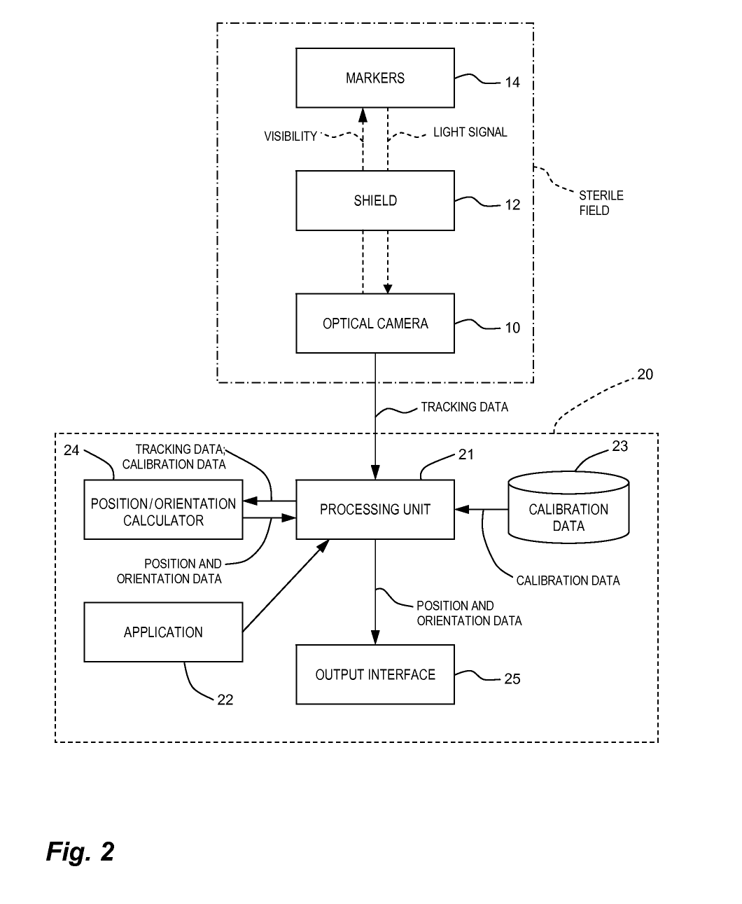

Referring to FIG. 2, the combination of the optical camera 10 and shield 12 detects markers 14 for navigation of a tool or object by navigation system 20. The markers 14 are secured to the tool or object being tracked.

The navigation system 20 receives tracking data for the markers 14 from the optical camera 10. The tracking data is typically a position value for each one of the markers 14. In order to navigate the position and orientation of an object, it may be required to track the position of at least three non-linear points (e.g., three markers 14) fixed to the object. Therefore, the navigation system 20 receives the tracking data for three different markers 14, and generates position and orientation data for an object from the three different markers 14.

In an embodiment, the navigation system 20 is part of a computer featuring a processing unit 21. The processing unit 21 is programmed with an application 22 to perform navigation in computer-assisted surgery. The processing unit 21 receives calibration data 23 based on the calibration of the optical camera 10 with the shield 12. More specifically, as the presence of the shield 12 alters the light signal received by the sensors of the optical camera 10, a calibration performed beforehand provides calibration data for the use of the optical camera 10 with the shield 12. The calibration of the optical camera 10 prior to its use, usually performed by a coordinate measuring machine (CMM), is described hereinafter.

A position/orientation calculator 24 receives the tracking data for the markers 14, and calculates a position and/or orientation of a tool or object (e.g., bone element). As is known for navigation systems in computer-assisted surgery, the calculation is based on geometrical data relating at least three of the markers 14 to the tool or object. The geometrical data may be obtained by well known ways, for instance using on-site calibration of tools, digitization of frames of reference for bones, preoperatively obtained digital models of tools, etc.

In order to take into account the effect of the shield 12 on the signal received by the camera 10, the position/orientation calculator 24 receives the shield/camera calibration data 23, so as to adjust the position of the markers 14 as a function of the presence of the shield 12. Therefore, with the tracking data of the markers 14, the shield/camera calibration data 23, and the geometrical data relating the markers 14 to an object or tool, the position/orientation calculator 24 calculates position and orientation data for the object or tool.

An output interface 25 outputs the position and/or orientation data in any suitable format, such as visual displays of navigated tools with respect to bones, numerical values associated with lengths and angles, or the like. The output interface 25 may also or alternatively output the position and orientation data to other applications of the computer-assisted surgery system. Moreover, the navigation system 20 may be part of computer-assisted surgery systems running applications such as orthopedic surgery, tool calibration, etc.

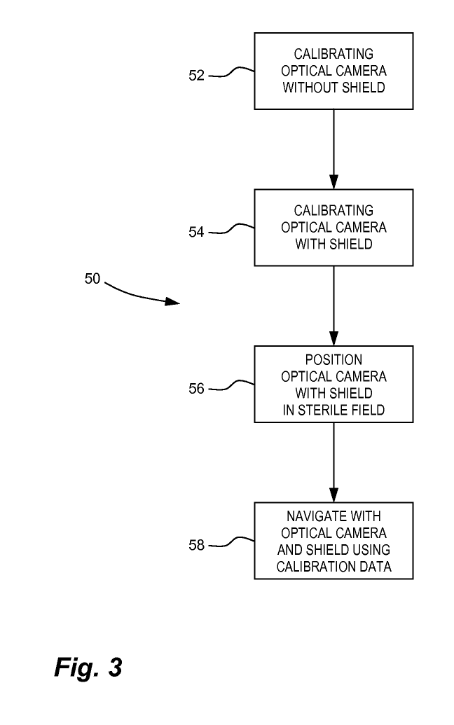

Referring to FIG. 3, a method for tracking tools and objects during computer-assisted surgery is generally illustrated at 50.

According to Step 52, the optical camera 10 is calibrated without the shield 12 (FIG. 1). In an embodiment, the calibration is performed using a coordinate measuring machine (CMM), as is known in the art. As the optical camera 10 may be used without the shield 12, it is preferable that a calibration be made for use of the optical camera 10 without the shield 12. The calibration data may be programmed into the optical camera 10 so that the tracking data output by the optical camera 10 is within the specified tolerances of the camera in terms of accuracy. Alternatively, the calibration data for the optical camera 10 may be provided to the navigation system 20. The calibration data 23 may be specific to a shield 12 and thus provided with the shield 12. If shields 12 having specifications within a controlled range, generic calibration data 23 may be provided in view of the use of the optical camera 10 with any one of the shields 12.

According to Step 54, the optical camera 10 is calibrated with the shield 12 (FIG. 1). The shield 12 is secured to the camera 10 in the known manner prior to the calibration being performed by the CMM. In an embodiment, calibration of the camera 10 is performed in a plurality of steps with different shields 12. In this embodiment, different shields are selected at the opposed limits of the acceptable range of densities for the polymeric material of the shield 12 so as to obtain calibration data taking into account the range of densities. The shield/camera calibration data is provided to the navigation system 20 (FIG. 2). It is also considered to program the optical camera 10 with the shield/camera calibration data so that the tracking data output by the optical camera 10 is within the specified tolerances of the camera 10 in terms of accuracy, when protected by the shield 12.

The Steps 52 and 54 are typically performed by the manufacturer of the optical camera 10, away from the surgical room. In an exemplary embodiment, the Steps 52 and 54 are performed prior to the use of the optical camera 10 in computer-assisted surgery, as these steps will ensure that the camera 10 outputs tracking data within specified tolerances. The Steps 52 and 54 may be performed in reversed order.

According to Step 56, the optical camera 10 equipped with the shield 12 in the known manner (FIGS. 1 and 2) is positioned within the sterile field for use with the navigation system 20 during computer-assisted surgery. Depending on its configuration, the optical camera 10 may be fully immersed into the sterile field, or partially therein. The shield 12 must be installed on the optical camera 10 when in the sterile field.

The shield 12 forms a screen between the patient and the optical camera 10, such that the patient cannot be contaminated by the optical camera 10 protected by the shield 12.

According to Step 58, the optical camera 10 equipped with the shield 12 may be used to navigate during computer-assisted surgery, with the shield/camera calibration data being used to correct the light signal detected by the camera 10, as altered when passing through the shield 12.

In order to ensure that the appropriate calibration data is used (i.e., with or without the shield 12), the navigation system 20 may prompt the operator to confirm whether a shield 12 is used.

In order to minimize the risk of errors, it is contemplated to use passive tracking with the material Scotchlite™, using a tracker configuration as described in United States Patent Application Publication No. 2007/0100325, by the present Assignee, the subject matter of which is incorporated herein by reference. When Scotchlite™ is used as retro reflective material during passive tracking, it has been shown to provide suitable results. It is also considered to use Scotchlite™ during CMM calibration as well.

1. A navigation system for tracking an object in a sterile field during surgery, comprising:
at least one marker adapted to be connected to the object in a known relation, the object being used in the sterile field during surgery;
an optical camera detecting a light signal from the at least one marker, the optical camera generating tracking data associated with a position of the at least one marker from the light signal;
a shield on the optical camera, the shield being positioned between the optical camera and the at least one marker during surgery to allow at least a portion of the optical camera to be within the sterile field;
calibration data associated with an effect of the shield on the light signal passing therethrough to be detected by the optical camera; and
a position and orientation calculator for calculating at least one of a position and an orientation of the object as a function of the tracking data, of the calibration data, and of the known relation between the object and the at least one marker.

2. The navigation system according to claim 1, wherein three of the markers are connected to the object, and the position and orientation calculator calculates at least one of the position and the orientation of the object with the tracking data of the three markers.

3. The navigation system according to claim 1, wherein the calibration data is pre-operative calibration data, the calibration data being loaded into the navigation system.

4. The navigation system according to claim 1, wherein the known relation between the at least one marker and the object is obtained intra-operatively.

5. The navigation system according to claim 1, wherein the shield separates an entirety of the optical camera from the object, whereby the optical camera is in the sterile field.

6. The navigation system according to claim 1, wherein the shield is a made of a molded polymer.

7. The navigation system according to claim 1, wherein the shield is transparent.

8. The navigation system according to claim 1, wherein the object is at least one of a bone and a surgical tool.

9. A method for tracking an object in computer-assisted surgery, comprising:
installing a shield on at least a portion of an optical camera, the shield affecting a light signal received by the optical camera;
positioning at least a portion of the optical camera within a sterile field with the object being in the sterile field and the shield being between the optical camera and the object; and
tracking the object as a function of a light signal from the object received by the optical camera after passing through the shield.

10. The method according to claim 9, further comprising calibrating the optical camera with the shield on the optical camera prior to tracking the object with the optical camera.

11. The method according to claim 10, wherein the calibrating is performed pre-operatively, away from the sterile field.

12. The method according to claim 9, wherein installing the shield comprises installing the shield on an entirety of the optical camera.

13. The method according to claim 12, wherein positioning the optical camera in the sterile field comprises fully positioning the optical camera in the sterile field.

14. The method according to claim 9, further comprising calibrating the optical camera without the shield using a coordinate measuring machine.
